(19) United States
(12) Patent Application Publication
(10) Pub. No.: US 2014/0294277 A1
(43) Pub. Date: Oct. 2, 2014

(54) IMAGE PROCESSING APPARATUS, IMAGE PROCESSING METHOD, AND STORAGE MEDIUM

(71) Applicant: CANON KABUSHIKI KAISHA, Tokyo (JP)

(72) Inventor: Shinya Katsumata, Tokyo (JP)

(73) Assignee: CANON KABUSHIKI KAISHA, Tokyo (JP)

(21) Appl. No.: 14/194,869

(22) Filed: Mar. 3, 2014

(30) Foreign Application Priority Data
Mar. 29, 2013 (JP) 2013-074862

(51) Int. Cl. G06T 7/00

(52) U.S. Cl. CPC G06T 7/0012
USPC 382/132

(57) ABSTRACT

An image processing apparatus for processing a radiation image obtained from a radiation detector capable of releasing or accumulating electric charges for each row, comprising: a target value setting unit configured to set a target value of each pixel on a correction target row in the radiation image based on pixel values on a row adjacent to the correction target row; a pixel selection unit configured to select an effective pixel on the correction target row based on a pixel value and the target value of each pixel on the correction target row; and a correction unit configured to derive a correction coefficient using a pixel value and the target value of the effective pixel and correct the correction target row based on the correction coefficient.

### BACKGROUND OF THE INVENTION

1. Field of the Invention

The present invention relates to an image processing apparatus, an image processing method, and a storage medium and, more particularly, to an image processing apparatus, an image processing method, and a storage medium, which perform, for an image obtained by imaging using a radiation detector formed by a plurality of pixels, correction of artifacts caused by a time difference, that is, a radiation detection delay between the time when radiation reaches the radiation detector and the time when the function of the radiation detector detects the radiation.

2. Description of the Related Art

Recently, a radiation imaging apparatus which uses a flat panel detector (to be referred to as an “FPD” hereinafter) made of a semiconductor material for medical image diagnosis and nondestructive inspection with radiation, particularly, X-rays has prevailed. In, for example, the field of medical image diagnosis, such radiation imaging apparatus is used as a digital imaging apparatus which can perform still image shooting such as general imaging, moving image shooting such as fluoroscopic imaging, and the like.

In general, such radiation imaging apparatus is configured to synchronize a radiation generation apparatus and an FPD so as to obtain the timing of starting radiation irradiation. However, since a connection apparatus for synchronizing the FPD and the radiation generation apparatus is generally required, the installation location may be limited.

To the contrary, in recent years, as described in Japanese Patent Laid-Open No. 2011-249891, a technique in which an FPD itself detects the start of radiation irradiation to perform imaging is known. In such apparatus, however, the timing when the FPD detects the start of radiation irradiation shifts from the timing of radiation irradiation. This may cause artifacts (to be also referred to as “detection delay artifacts” hereinafter) in a radiation image.

As a method of correcting the detection delay artifacts, true pixel values (to be also referred to as “true values” hereinafter) on a row of the FPD where the detection delay artifacts have occurred are derived using pixel values on the row where the detection delay artifacts have occurred and pixel values on its adjacent row. If, however, a true value is simply derived using pixel values on the row of the FPD where the detection delay artifacts have occurred and pixel values on its adjacent row, an error becomes large due to the influence of the edges of an object and the like.

The present invention has been made in consideration of the above problem, and provides a technique of correcting detection delay artifacts while reducing the influence of an object.

### SUMMARY OF THE INVENTION

According to one aspect of the present invention, there is provided an image processing apparatus for processing a radiation image obtained from a radiation detector capable of releasing or accumulating electric charges for each row, comprising: a target value setting unit configured to set a target value of each pixel on a correction target row in the radiation image based on pixel values on a row adjacent to the correction target row; a pixel selection unit configured to select an effective pixel on the correction target row based on a pixel value and the target value of each pixel on the correction target row; and a correction unit configured to derive a correction coefficient using a pixel value and the target value of the effective pixel and correct the correction target row based on the correction coefficient.

Further features of the present invention will become apparent from the following description of exemplary embodiments (with reference to the attached drawings).

### BRIEF DESCRIPTION OF THE DRAWINGS

FIG. 1A is a view showing an example of the configuration of a radiation imaging system according to the first embodiment;

FIG. 1B is a block diagram showing an example of the functional arrangement of an image processing apparatus according to the first embodiment;

FIG. 2 is a circuit diagram showing the hardware arrangement of a radiation detector;

FIG. 3 is a flowchart illustrating the procedure of artifact correction processing executed by the image processing apparatus according to the first embodiment;

FIG. 4 is a timing chart showing a procedure of driving a sensor array for detecting radiation;

FIG. 5 is a view showing a detection delay artifact image and its pixel values in an electric charge release method according to the first embodiment;

FIG. 6 is a view for explaining target value setting processing according to the first embodiment;

FIG. 7 is a timing chart showing a procedure of driving a sensor array for detecting radiation;

FIG. 8 is a view showing a detection delay artifact image and its pixel values in an electric charge release method according to the second embodiment;

FIG. 9 is a flowchart illustrating the procedure of pixel selection processing executed by the image processing apparatus according to the first embodiment;

FIG. 10 is a flowchart illustrating the procedure of temporary correction coefficient deriving processing executed by the image processing apparatus according to the first embodiment;

FIG. 11 is a view for explaining a pixel value sort according to the first embodiment;

FIG. 12 is a view for explaining processing of selecting an effective pixel by a pixel selection unit according to the first embodiment;

FIG. 13 is a view for explaining processing of selecting an ineffective pixel by the pixel selection unit according to the first embodiment;

FIG. 14 is a graph showing the frequency distribution of temporary correction coefficients according to the first embodiment; and

FIG. 15 is a timing chart showing a procedure of driving a sensor array for detecting radiation.

### DESCRIPTION OF THE EMBODIMENTS

An exemplary embodiment(s) of the present invention will now be described in detail with reference to the drawings. It should be noted that the relative arrangement of the components, the numerical expressions and numerical values set forth in these embodiments do not limit the scope of the present invention unless it is specifically stated otherwise.

### First Embodiment

### Configuration of Radiation Imaging System

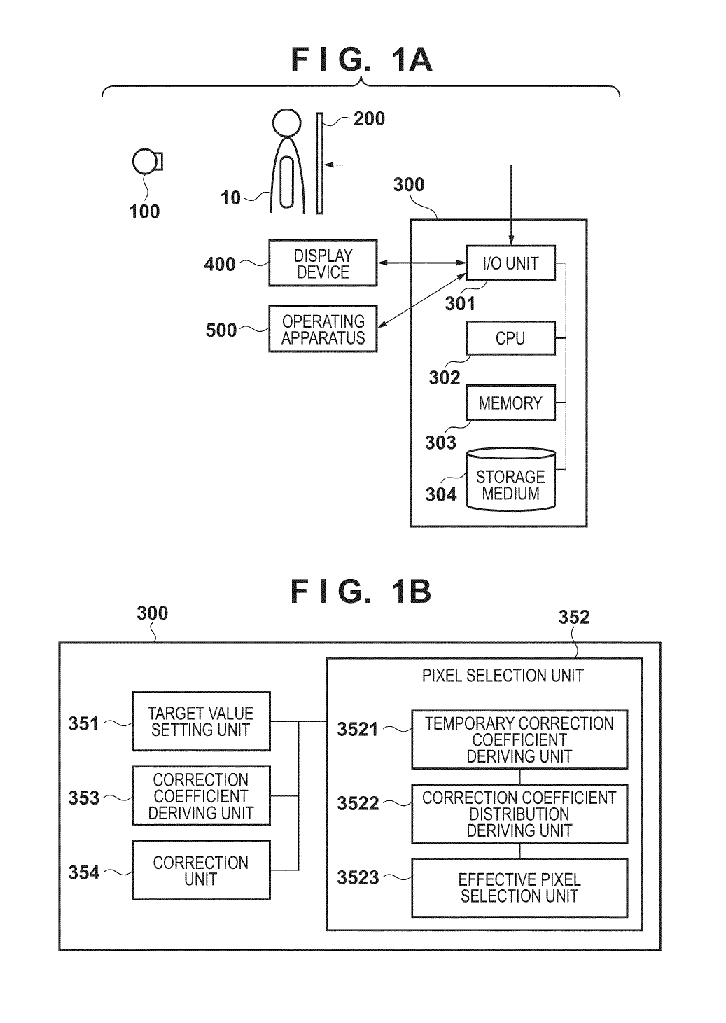

An example of the configuration of a radiation imaging system according to the first embodiment will be described first with reference to FIG. 1A. The radiation imaging system includes a radiation generation apparatus 100, a radiation detector 200, an image processing apparatus 300, a display device 400, and an operating apparatus 500.

An object 10 is located between the radiation generation apparatus 100 and the radiation detector 200. In this state, the radiation generation apparatus 100 performs radiation irradiation toward the radiation detector 200. The radiation detector 200 detects the radiation to generate a radiation image, and transmits the generated radiation image to the image processing apparatus 300 connected to the radiation detector 200.

The image processing apparatus 300 includes an I/O unit 301, a CPU 302, a memory 303, and a storage medium 304. The I/O unit 301, for example, transmits/receives various kinds of data. The CPU 302 controls the operation of the image processing apparatus 300. The memory 303 reads and writes programs, data, and the like calculated by the CPU 302. The storage medium 304 stores radiation image data having undergone image processing and the like. The image processing apparatus 300 is connected to the display device 400 which displays a processing result, a radiation image, and the like, and the operating apparatus 500 which is used to accept a user operation.

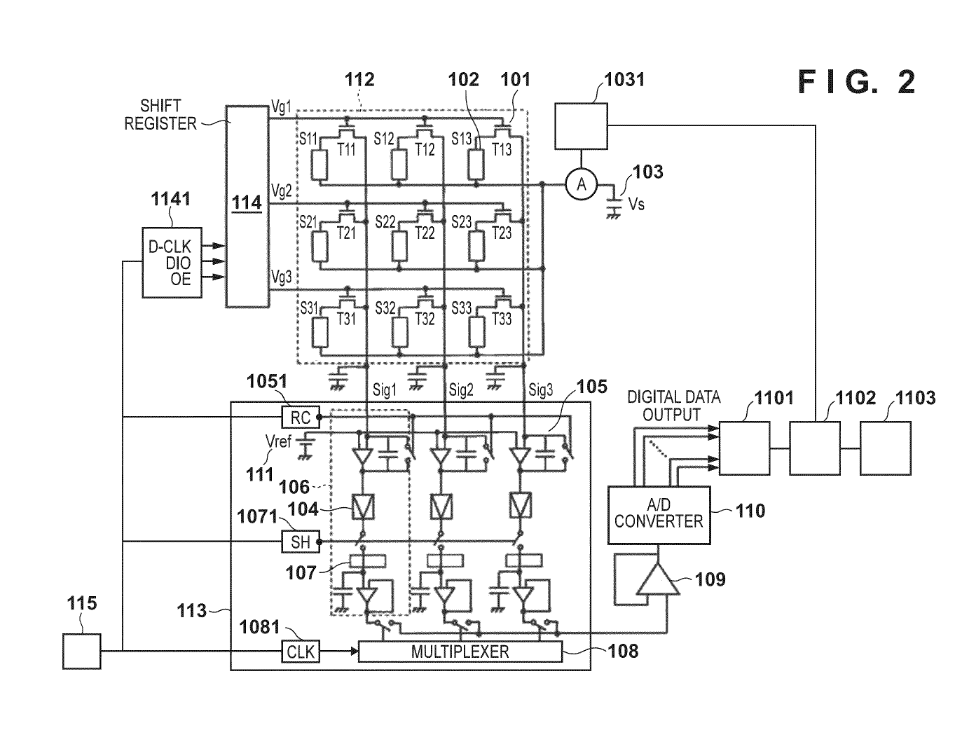

An operator inputs an operation for starting imaging using the operating apparatus 500. This operation indicates a general operation of exchanging information for imaging preparation between the radiation detector 200 and the image processing apparatus 300. The radiation generation apparatus 100 generates radiation so that all the radiation detection elements of the radiation detector 200 are irradiated with radiation. Upon receiving radiation, the radiation detector 200 which includes a circuit equivalent to that shown in FIG. 2 (to be described later) can accumulate or release electric charges for each row of an FPD, and transmits, to the image processing apparatus 300, the coordinates of a row (to be referred to as a “detection row” hereinafter) of the FPD where the irradiated radiation has been detected, and image data (to be referred to as an “artifact image” hereinafter) obtained by converting radiation received by each radiation detection element into a digital signal.

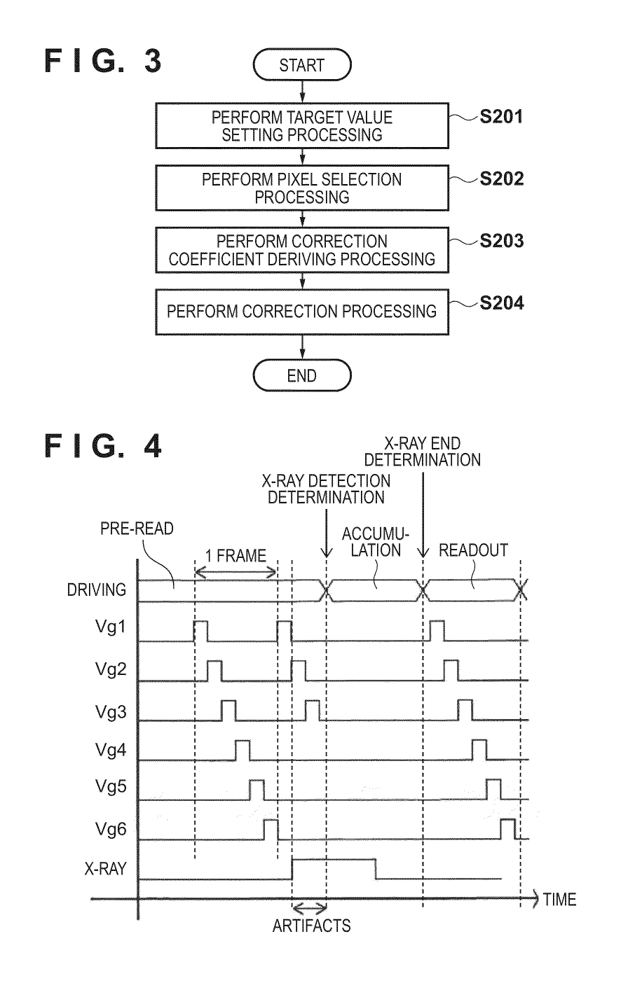

The image processing apparatus 300 performs detection delay artifact correction processing (to be described later with reference to FIG. 3) for each row from the detection row of the artifact image received from the radiation detector 200, generates an image (to be referred to as a “corrected image” hereinafter) having undergone the detection delay artifact correction processing, and transmits the generated image to the display device 400. Note that the image processing apparatus 300 may transmit the corrected image having further undergone image processing such as known tone processing and frequency processing to the display device 400. The display device 400 displays, to the operator, the corrected image received from the image processing apparatus 300.

<Functional Arrangement of Image Processing Apparatus 300>

FIG. 1B is a block view showing the functional arrangement of the image processing apparatus 300. The image processing apparatus 300 processes a radiation image obtained from the radiation detector 200 capable of releasing or accumulating electric charges for each row. The image processing apparatus 300 includes a target value setting unit 351, a pixel selection unit 352, a correction coefficient deriving unit 353, and a correction unit 354. The pixel selection unit 352 includes a temporary correction coefficient deriving unit 3521, a correction coefficient distribution deriving unit 3522, and an effective pixel selection unit 3523.

Based on pixel values on a correction target row of the radiation image where artifacts have occurred and pixel values on a row adjacent to the correction target row, the target value setting unit 351 sets true pixel values on the correction target row as target values.

Based on the pixel values and target values on the correction target row, the pixel selection unit 352 selects, as an effective pixel, a pixel on the correction target row, which suffers a small influence of the object. The correction coefficient deriving unit 353 derives a correction coefficient for generating a corrected image by using the pixel value and target value of the effective pixel. The correction unit 354 generates a corrected image by correcting artifacts in the radiation image using the pixel value and target value of the effective pixel.

The temporary correction coefficient deriving unit 3521 derives temporary correction coefficients based on the pixel values and target values on the correction target row. The correction coefficient distribution deriving unit 3522 obtains the distribution of the temporary correction coefficients. Based on the distribution of the temporary correction coefficients, the effective pixel selection unit 3523 selects, as an effective pixel, a pixel on the correction target row, which suffers a small influence of the object.

The hardware arrangement of the radiation detector 200 will be described with reference to FIG. 2. The radiation detector is, for example, a portable radiation detector including two-dimensionally arrayed radiation sensors, their peripheral circuits, and a battery in an almost cubic housing. The radiation detector 200 includes a fluorescent material for converting radiation into visible light, and a sensor array 112. The sensor array 112 is formed by arraying, in a matrix pattern, pixels each including a photoelectric conversion element 102 for converting visible light into an electric signal and a TFT 101 serving as a switching element. In the example shown in FIG. 2, nine photoelectric conversion elements 102 or S11 to S33 and nine TFTs 101 or T11 to T33 are arrayed in a 3×3 matrix for descriptive convenience. In fact, it is desirable that several thousand pixels are vertically and horizontally arrayed.

One end of the photoelectric conversion element 102 is connected with the corresponding TFT 101 and the other end of the photoelectric conversion element 102 is connected with a feeder line which connects the photoelectric conversion element 102 to a bias supply 103. The gate of each TFT 101 is connected to a vertical driving circuit via a corresponding one of row selection lines Vg1 to Vg3 which are commonly used for respective rows. A conductive voltage from the shift register 114 of the vertical driving circuit controls ON/OFF of each TFT 101. The source or drain of each TFT 101 is connected to a corresponding one of column signal lines Sig1 to Sig3. If the TFT 101 is turned on, the electric signal of the corresponding photoelectric conversion element 102 is read out via the corresponding column signal line. A readout circuit 113 amplifies the readout electric charges. In the readout circuit 113, an amplification circuit 106 which includes an integration amplifier 105 connected to an amplifier reference supply 111, a variable gain amplifier 104, and a sample/hold circuit 107 is provided on each row. Each amplification circuit 106 is connected to a multiplexer 108 which performs parallel-serial conversion. An output from the multiplexer is input to an A/D converter 110 via an output buffer amplifier 109, and converted into a digital value by the A/D converter 110. Under the control of a processing circuit 1101, the digital value is stored in a memory 1102 as radiation image data. A communication circuit 1103 transmits the radiation image data to the image processing apparatus 300 by wired or wireless connection.

A driving control unit 1141 of the vertical driving circuit controls the input of the shift register 114. The shift register 114 generates a driving clock D-CLK indicating a driving timing, driving data DIO indicating a driving method, and an output effective signal OE for collectively controlling an output, which control the ON/OFF timings and order of the TFTs 101. A signal RC from an amplification control unit controls the operation timing of the integration amplifier. A signal SH from a sample/hold control unit 1071 controls a sample/hold timing. A signal CLK from a parallel-serial conversion control unit 1081 controls parallel-serial conversion by the multiplexer 108. The driving control unit 1141, amplification control unit, sample/hold control unit 1071, and parallel-serial conversion control unit 1081 are connected to an imaging control unit 115 and controlled by it.

The feeder line which connects the bias supply 103 and the photoelectric conversion elements 102 is connected with an ammeter A which measures a current flowing through the feeder line. The ammeter A is connected to an irradiation determination circuit 1031 which determines, based on the current amount measured by the ammeter A, that radiation irradiation has been performed.

Radiation irradiation causes the photoelectric conversion elements 102 to generate electric charges. In this case, if the TFTs 101 are OFF, a current accordingly flows through the feeder line. Furthermore, if the TFTs 101 are turned on after radiation irradiation is performed and the photoelectric conversion elements 102 generate electric charges, electric signals corresponding to the electric charges are output. As a result, a current flows through the feeder line to compensate for the output electric charges. Measuring the current enables detection of radiation irradiation. A current which flows through the feeder line when the TFT is turned on is larger than that which flows through the feeder line when the TFT is OFF. This is advantageous in early detection of radiation irradiation.

The irradiation determination circuit 1031 outputs, as determination timing data, the row number of a row selection line which is ON when it is determined that radiation irradiation has been performed. Radiation detection timing data and time-series data of the current measured by the ammeter are input to the memory 1102, associated with the radiation image data, and transmitted by the communication circuit 1103.

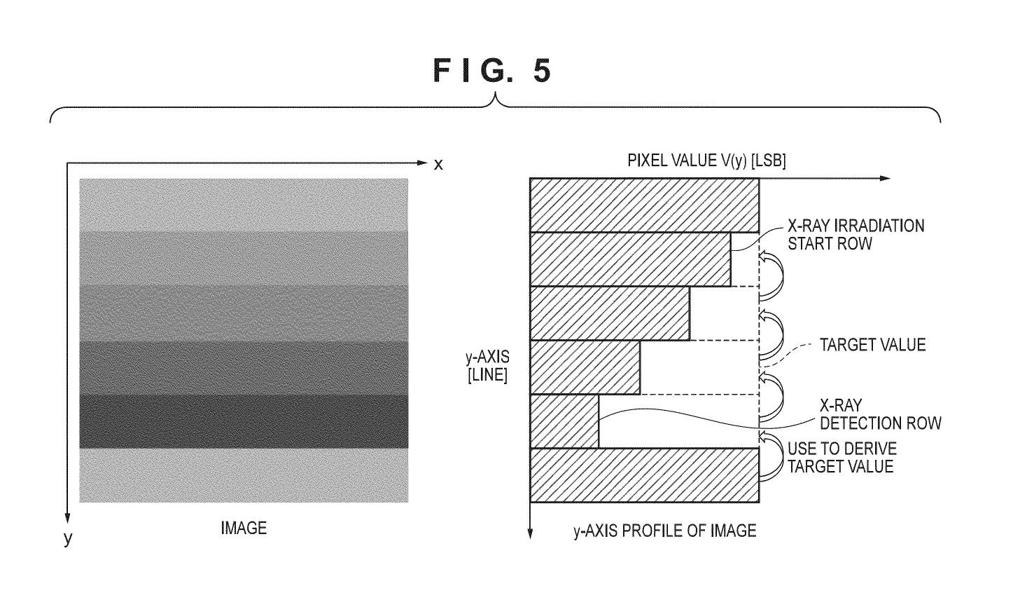

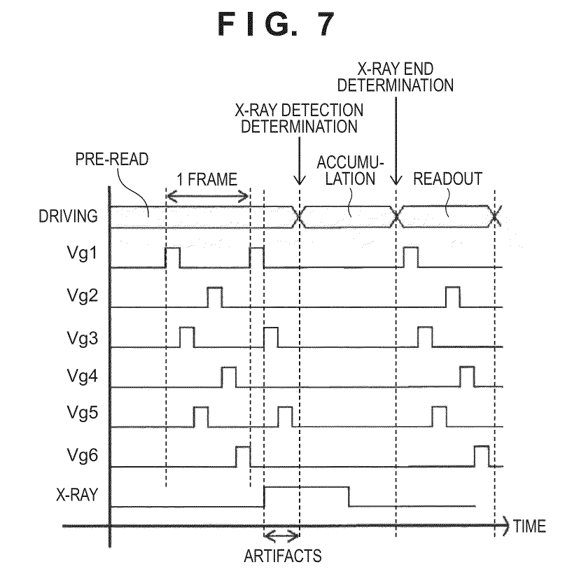

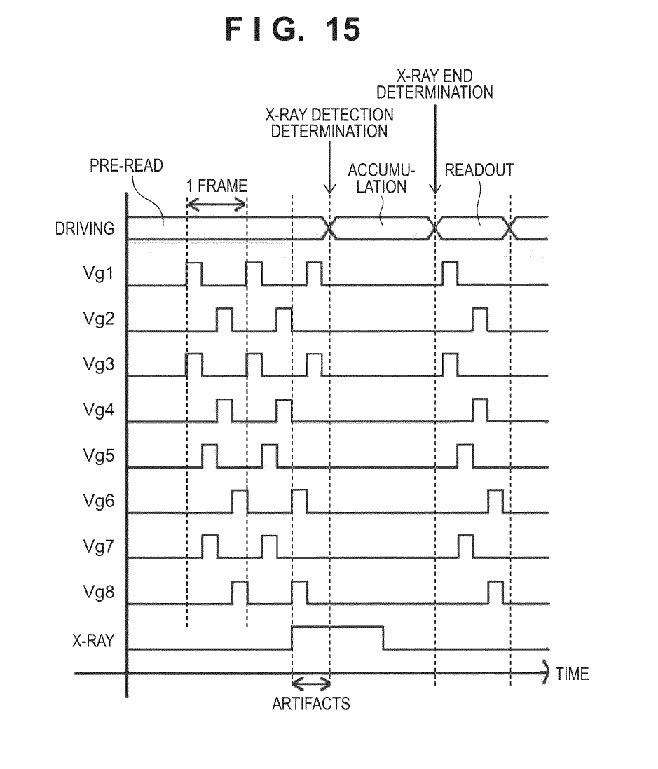

A method of driving the sensor array 112 for detecting radiation will be described with reference to timing charts shown in FIGS. 4, 7, and 15. In each of the timing charts, the abscissa represents the time and the ordinate represents a driving phase as “driving”, the timings of applying a conductive voltage to respective row selection lines Vgi, and an X-ray irradiation timing. In the examples shown in FIGS. 4 and 7, six row selection lines Vg are included. In the example shown in FIG. 15, eight row selection lines Vg are included. However, the number of row selection lines can be changed depending on implementation of the sensor array 112.

In the example shown in FIG. 4, a conductive voltage is applied at exclusive timings, that is, in the order of Vg1, Vg2, Vg3, . . . . When application of the conductive voltage up to the last row selection line Vg6 is finished and a readout operation for one frame is complete, the conductive voltage is applied again from the row selection line Vg1. This driving operation is represented by “PRE-READ”. At this time, X-ray irradiation is performed, and a larger current flows through the feeder line.

The ammeter A repeatedly measures the current at predetermined intervals. The irradiation determination circuit 1031 acquires a digital measurement value, and repeatedly performs determination processing for comparing, with a threshold, a value obtained by performing difference processing with an immediately preceding frame or a preceding frame and the like. If the threshold is exceeded, it is determined that X-ray irradiation has been performed. After that, the signal OE is input to the shift register 114, and all the TFTs 101 are turned off. This state is represented by “accumulation”. After that, the shift register 114 sequentially reads out electric signals and the readout circuit 113 amplifies the electric signals, thereby obtaining radiation image data.

In the example shown in FIG. 7, a conductive voltage is applied to the alternate row selection lines Vg1, Vg3, Vg5, . . . . The vertical driving circuit controls so that adjacent rows are not successively turned on.

In the example shown in FIG. 15, it is controlled to apply a voltage to the row selection lines Vg1 and Vg3 at a given timing, the row selection lines Vg5 and Vg7 at the next timing, the row selection lines Vg2 and Vg4 at the next timing, and the row selection lines Vg6 and Vg8 at the next timing. That is, the vertical driving circuit controls to turn on the TFTs on each row in a predetermined order so as not to simultaneously turn on the TFTs 101 on adjacent rows while simultaneously turning on the TFTs 101 on a plurality of rows.

<Principle of Generation of Detection Delay Artifacts>

The principle of generation of detection delay artifacts will be described next. While X-ray irradiation is not performed, the FPD generally drives the circuit to release electric charges for each row (or column) in order to prevent a dark current from remaining in the capacitor of each pixel. An FPD of a type which does not detect the start of X-ray irradiation by itself stops releasing electric charges to move on to an electric charge accumulation operation by obtaining in advance an X-ray irradiation start timing from the X-ray generation apparatus.

On the other hand, an FPD (the radiation detector 200) of a type according to the embodiment which detects the start of X-ray irradiation by itself stops releasing electric charges to move on to an electric charge accumulation operation upon detecting the start of X-ray irradiation by detection processing (a detection method varies depending on an FPD, in which, for example, electric charges within a pixel are read out and determination is made based on the amount of electric charges) within the FPD, as described above. Note that a row of the FPD, for which electric charges have been released last, is a “detection row”.

As described above, there is a time lag from when X-ray irradiation is actually performed until the FPD detects the start of X-ray irradiation. During the time lag, electric charges accumulated in a pixel by X-ray irradiation are unwantedly released. Some pixel values of an image decrease by the released electric charges, resulting in artifacts in the image.

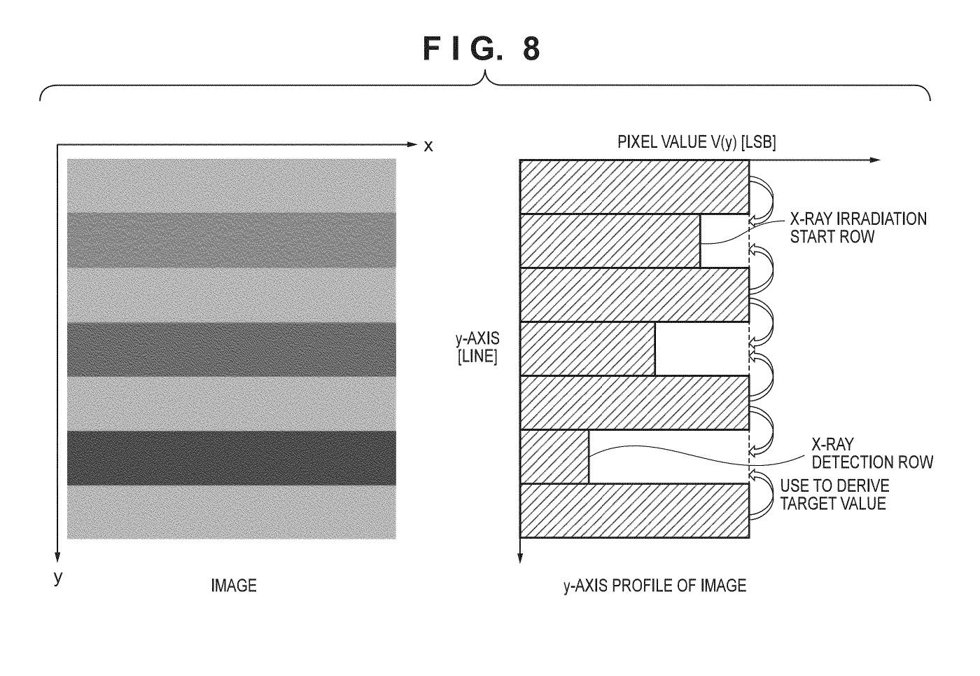

Each of FIGS. 5 and 8 shows the neighborhood of an artifact occurrence portion of an FPD of a type which releases electric charges for each row. The difference between FIGS. 5 and 8 is that the FPD shown in FIG. 5 sequentially releases electric charges from the upper portion like the driving operation shown in FIG. 4 while the FPD shown in FIG. 8 adopts a scheme of releasing electric charges on alternate rows from the upper row like the driving operation shown in FIG. 7. Whether artifacts continuously or discretely occur depends on the method of releasing electric charges of the FPD. A description will be provided with reference to FIG. 5 in the first embodiment while a description will be provided with reference to FIG. 8 in the second embodiment.

<Concept of Correction of Detection Delay Artifacts>

The concept of correction of detection delay artifacts according to the embodiment will be described next. As described above, on a row where artifacts occur, not all pixel values are lost, and only some electric charges (pixel values) accumulated from the start of X-ray irradiation until electric charges are released are lost.

To correct detection delay artifacts, therefore, it is only necessary to compensate for a pixel value V_{A}(x, y) lost by release of electric charges. Thus, it is possible to obtain a true value V′(x, y) when no artifact occurs by calculating the sum of a pixel value V(x, y) on an artifact occurrence row and the lost pixel value V_{A}(x, y), as given by:

V′(x,y)=V(x,y)+V_{A}(x,y) (1)

The lost pixel value V_{A}(x, y) can be represented by the product of a value obtained by subtracting an accumulation component V_{dark}(y) due to a dark current from the true value V′(x, y) and a time ratio R(y) between the total X-ray irradiation time and the time from when X-ray irradiation is performed until the electric charges of a corresponding pixel are released, given by:

V_{A}(x,y)=R(y)·(V′(x,y)−V_{dark}(y)) (2)

Note that release of electric charges is performed for each row in the y-axis direction. Therefore, R(y) and V_{dark}(y) depend on not the x direction but only the y direction.

Note that the lost pixel value V_{A}(x, y) is the difference between the true value V′(x, y) and the pixel value V(x, y) on the artifact occurrence row, given by:

V_{A}(x,y)=V′(x,y)−(x,y)=R(y)·(R′(x,y)−V_{dark}(y)) (3)

Since the true value V′(x, y) is an unknown value, replacing the true value V′(x, y) by a target value Vo(x, y) derived from an adjacent pixel where no artifacts have occurred yields:

V_{O}(x,y)−(x,y)=R(y)·(V_{O}(x,y)−V_{dark}(y)) (4)

Deriving of the target value Vo(x, y) will be described in target value setting processing in step S201 of FIG. 3 (to be described later).

Modifying the right-hand side of equation (4) yields:

V_{O}(x,y)−V(x,y)=A(y)·Vₒ(x,y)+B(y) (5)

Note that A(y) is equal to R(y) and B(y) is equal to −R(y)·V_{dark}(y). Equation (5) is a linear equation. By means of simultaneous equations obtained by substituting the values of different pixels on the yth row, it is possible to derive the coefficients A(y) and B(y). In this case, using the target value Vo(x, y) simply derived from an adjacent pixel instead of the true value V′(x, y) may result in a large error.

To avoid this situation, it is necessary to use only a pixel of the target value Vo(x, y) close to the true value V′(x, y). Using only an effective pixel makes it possible to obtain the coefficients A(y) and B(y) with high reliability for each row. Note that processing of determining whether the target value Vo(x, y) is close to the true value V′(x, y), and selecting an effective pixel will be described in pixel selection processing in step S202 of FIG. 3 (to be described later). Finally, it is possible to obtain a corrected value Vc(x,y) using equation (6) derived from equations (1) and (5).

V_{C}(x,y)=V(x,y)+A(y)·V_{O}(x,y)+B(y) (6)

<Detection Delay Artifact Correction Processing by Image Processing Apparatus 300>

The procedure of detection delay artifact correction processing executed by the image processing apparatus 300 according to the first embodiment will be described below with reference to a flowchart shown in FIG. 3.

An overview of the whole processing will be explained first and details of each process will be described later. In step S201, the target value setting unit 351 derives estimated true pixel values (target values) on a detection delay artifact occurrence row from an artifact image and a detection row.

In step S202, the pixel selection unit 352 selects a pixel (effective pixel) which suffers a small influence of an increase/decrease in pixel value due to the influence of an object based on the target values derived in step S201 and the artifact image.

In step S203, the correction coefficient deriving unit 353 extracts only the effective pixel derived in step S202, and derives a correction coefficient based on the effective pixel. In step S204, the correction unit 354 creates a corrected image from the artifact image using the correction coefficient derived in step S203.

A detailed description will be provided by exemplifying the radiation detector 200 which sequentially releases electric charges from upper to lower portions of the image as shown in FIG. 5. In the case of the radiation detector 200 shown in FIG. 5, normal pixels free of detection delay artifacts exist from a row next to the detection row. However, detection delay artifacts have occurred in all adjacent pixels on a row before the detection row. It is, therefore, necessary to sequentially perform processing using the result of artifact correction from the detection row to preceding rows. Assume that detection delay artifact correction is performed from the detection row shown in FIG. 5 for each row in the upper direction.

<Target Value Setting Processing: S201>

In step S201, the target value setting unit 351 derives the target value Vo(x, y) to be used instead of the true value V′(x, y) according to the above-described equation. Since rows succeeding the artifact row as a correction target have normal pixel values, a pixel value on the next row may be set as a target value, an average value obtained by using a plurality of rows including the next row and subsequent rows may be set as a target value, or extrapolation prediction may be performed. As an extrapolation prediction method, linear prediction may be used, or an interpolation method which uses the Burg method, prediction by a multidimensional polynomial, or the like, and takes a frequency into consideration may be used. Frequency reduction processing of reducing frequency components which interfere with artifacts may be performed.

In linear prediction, if a pixel value is small and a noise amount is large, an error between the true value and the target value becomes large. Therefore, the noise amount is compared with a gradient obtained by linear prediction. If the noise amount is small, the result of linear prediction is adopted; otherwise, the result of linear prediction is not adopted.

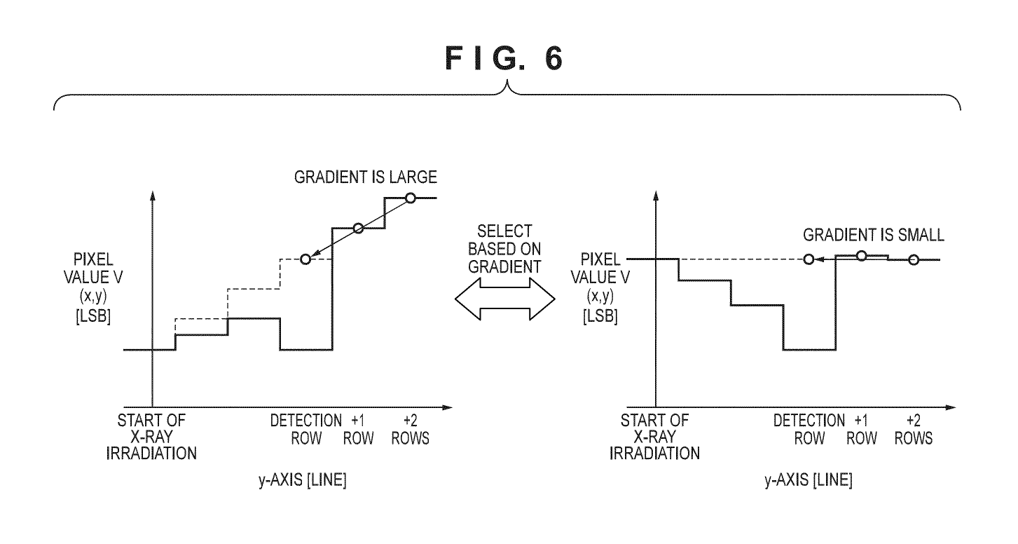

As shown in FIG. 6, for example, a gradient value is derived from the difference between a pixel value on a row (“+1 row”) next to the artifact row (detection row) and that on the second succeeding row (“+2 rows”). The derived gradient value is compared with the standard deviation of the noise of the pixel value on the row (“+1 row”) next to the artifact row. If the gradient value is larger, a value obtained by performing linear interpolation based on the gradient is set as a target value. On the other hand, if the noise amount is larger, the average value of the values on the “+1 row” and “+2 rows” of FIG. 6 is set as a target value. Note that the noise amount can be derived by, for example, performing imaging in advance without arranging the object and obtaining the relationship between a pixel value and a standard deviation.

<Pixel Selection Processing: S202>

In step S202, the pixel selection unit 352 selects a pixel (effective pixel) which suffers a small influence of an increase/decrease in pixel value due to the influence of the object based on the target value of each pixel derived in step S201 and the artifact row of the artifact image. The pixel selection unit 352 excludes a pixel in which there is an error between the target value and the true value due to a difference (step or the like) of the object reflected on the pixel, and selects a pixel in which the target value is close to the true value.

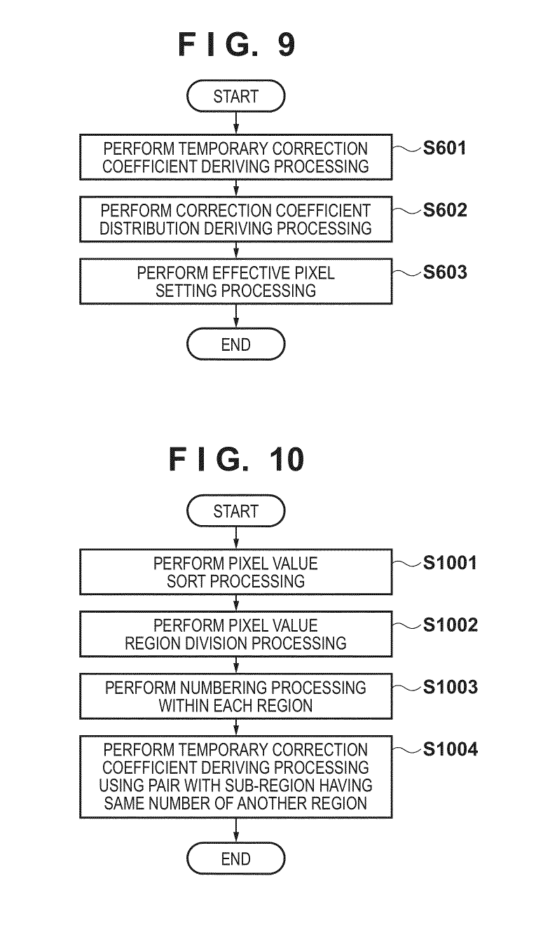

The procedure of the pixel selection processing in step S202 will be described with reference to a flowchart shown in FIG. 9. The procedure of the overall processing will be explained first. In step S601, the temporary correction coefficient deriving unit 3521 derives each temporary correction coefficient of equation (5) from the pixel values and the target values on the artifact row by solving the simultaneous equations.

In step S602, the correction coefficient distribution deriving unit 3522 creates the frequency distribution of the temporary correction coefficients derived in step S601. Finally, in step S603, the effective pixel selection unit 3523 selects an effective pixel on the assumption that a pixel in which the target value does not suffer the influence of the step of the object has a larger number of temporary correction coefficients (a higher appearance frequency).

An overview of each process will be described below. The processing procedure of the temporary correction coefficient deriving unit 3521 will be explained with reference to a flowchart shown in FIG. 10. The procedure of the overall processing will be described first. Pixel sort processing of sorting the pixels based on the input target values of the respective pixels derived in step S201, and accordingly sorting the corresponding pixel values on the artifact row is performed (step S1001). Pixel value region division processing of dividing a region based on the sorted target values and the pixel values on the artifact row is performed (step S1002). Numbering processing which numbers sub-regions in the respective regions is executed (step S1003). Temporary correction coefficient deriving processing is performed based on the determined number using a pair with a sub-region having the same number of another region (step S1004), thereby completing the temporary correction coefficient deriving processing.

Practical processing will be explained next.

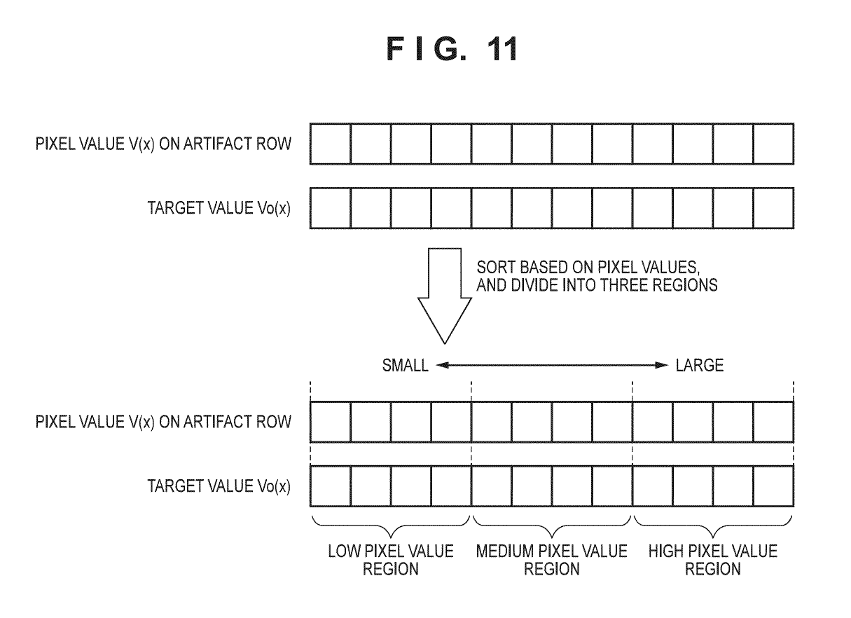

If a pixel value and a target value are close to each other when obtaining temporary correction coefficients from the simultaneous linear equations given by equation (5), the target value and the pixel value may become equal to each other. In this case, it is impossible to obtain a solution. To solve this problem, the pixels are sorted based on the pixel values/target values, as shown in FIG. 11. Note that whether to exclude two pixels used to obtain the simultaneous equations cannot be determined by one coefficient. It is, therefore, necessary to derive coefficients from two different pairs of pixels.

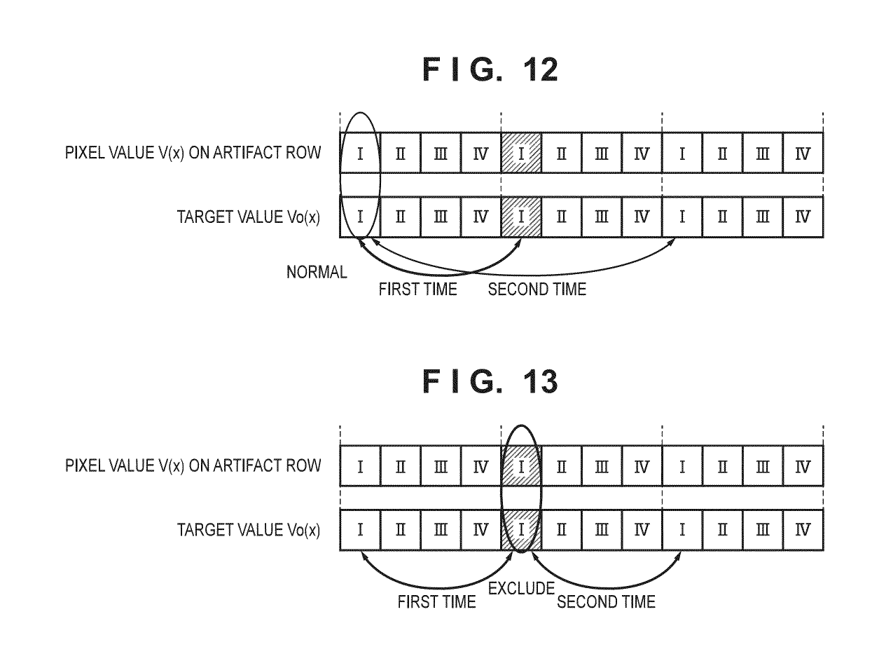

The sorted pixels are grouped into three regions (a low pixel value region, a medium pixel value region, and a high pixel value region), and a coefficient is derived twice for each pixel using corresponding pixels in the other two regions. In this case, to determine pairs for calculating coefficients in the three pixel value regions, the respective pixels within each region are numbered as shown in FIG. 12, so that each pixel can be paired with respective pixels having the same number in the other two regions.

As preprocessing for determining whether the pixel is an effective pixel, the distribution of the temporary coefficients is derived. Since most edges of the object occupy in the horizontal direction at a low probability, a threshold range for setting, as normal pixels, pixels with coefficients close to a coefficient with the maximum value of the distribution of the temporary coefficients and excluding the remaining pixels is determined. If a pixel has a value which excludes two derived coefficients, the pixel is determined as an ineffective pixel to be excluded.

<Temporary correction Coefficient Deriving Processing: S601>

As shown in FIGS. 10 and 11, the pixels are sorted according to the magnitude relationship between the pixel values of the target values Vo(x), and grouped into three regions, that is, a low pixel value region, a medium pixel value region, and a high pixel value regions, so that the respective regions include the same number of pixels. At this time, depending on the total number of pixels, some pixels may remain, but the number of such pixels is very small in terms of the total number of pixels on the whole row. Such remaining pixels may be, for example, excluded from the subsequent correction coefficient deriving processing in step S203.

The pixel group of each of the three regions undergoes numbering processing, as shown in FIG. 12. Each of the numbered pixels is paired with a pixel in a region different from the self region, thereby deriving a temporary correction coefficient according to simultaneous equations given by equation (4). As shown in FIGS. 12 and 13, it is possible to extract a plurality of pairs (two pairs) for one pixel, and derive two pairs of temporary correction coefficients A(y) and B(y) corresponding to the pixel pairs.

<Correction Coefficient Distribution Deriving Processing: S602>

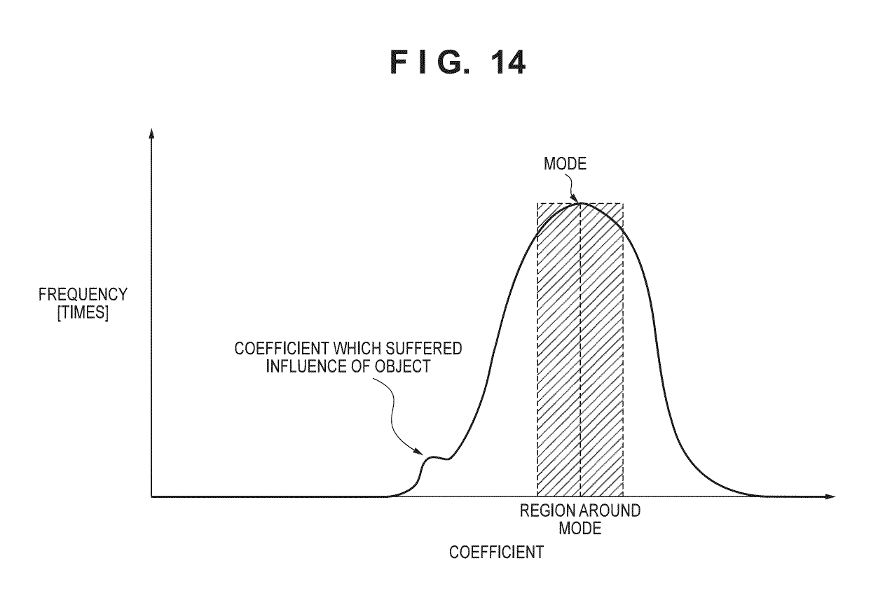

The correction coefficient distribution deriving unit 3522 creates the frequency distribution of the temporary correction coefficients derived in step S601. A frequency profile as shown in FIG. 14 is derived from all the temporary correction coefficients obtained in step S601. The correction coefficients used at this time may be A(y) or B(y) of equation (3). When creating the profile, if the number of temporary correction coefficients derived in step S601 is small, the profile becomes discrete or an unexpected bias occurs. Therefore, smoothing processing may be performed by low-pass filtering or a moving average method.

Since a true correction coefficient should take the same value on one row, the profile converges to one feature value (for example, a mode), as shown in FIG. 14. If the influence of the object is exerted, the influence appears at a position shifted from the highest peak, as represented by a peak of the coefficient which has suffered the influence of the object in FIG. 14.

<Effective Pixel Setting Processing: S603>

The effective pixel selection unit 3523 derives the mode as shown in FIG. 14 from the frequency profile derived in step S602. If both the two pairs of the temporary correction coefficients obtained in step S601 fall outside a given threshold range from the mode, the pixel is determined as an ineffective pixel. If at least one of the pairs falls within the threshold range, the pixel is determined as an effective pixel. Furthermore, it may be further configured to determine, as an ineffective pixel, a pixel which has a pixel value equal to or larger than a saturation pixel value and does not satisfy the linearity between the pixel value and the radiation dose of the radiation detector 200.

The threshold range obtained from the mode may be a fixed distance range from the mode, or a range within which a given percentage of the total number of coefficients used for the profile falls. More specifically, a range within which 20% of the total number of pixels with the mode as the center fall is set as a threshold range.

In the example of FIG. 12, pairs for deriving temporary correction coefficients of the Ith pixel in the low pixel value region are shown. In this example, if the Ith pixel in the medium pixel value region is a pixel which suffers a strong influence of the object, a pair with a corresponding pixel in the medium pixel value region for deriving temporary correction coefficients of the Ith pixel in the low pixel value region falls outside the threshold range and a pair with a corresponding pixel in the high pixel value region falls within the threshold range, and thus the pixel can be determined as an effective pixel.

In the example of FIG. 13, pairs for deriving temporary correction coefficients of the Ith pixel in the medium pixel value region are shown. In this example, if the Ith pixel in the medium pixel value region is a pixel which suffers a strong influence of the object, the temporary correction coefficients derived using the two pairs with corresponding pixels in other regions fall outside the threshold range, and thus the pixel can be determined as an ineffective pixel. The processing shown in FIG. 9 ends, thereby terminating the pixel selection processing in step S202 of FIG. 2.

<Correction Coefficient Deriving Processing: S203>

In step S203, the correction coefficient deriving unit 353 derives correction coefficients using the target values on the artifact row derived in step S201 and the effective pixel derived in step S202. To derive correction coefficients, equation (5) is used. As a deriving method, the least squares method using only the effective pixel derived by the pixel selection processing in step S202 is performed. In performing the least squares method, a robust estimation method (such as M-estimation, least median of squares, and RANSAC) may be used to improve the accuracy.

<Correction Processing: S204>

In step S204, the correction unit 354 uses coefficients (A(y) and B(y)) on each row derived in step S203 to derive a corrected value Vc(x, y) based on a pixel value V(x, y) and target value Vo(x, y) on the artifact row according to equation (6), and generates a corrected image based on the corrected values. Each process in FIG. 3 then ends.

As described above, according to this embodiment, there is provided an image processing apparatus for processing a radiation image obtained from a radiation detector capable of releasing or accumulating electric charges for each row, comprising a target value setting unit configured to set a target value of each pixel on a correction target row in the radiation image based on pixel values on a row adjacent to the correction target row, a pixel selection unit configured to select an effective pixel on the correction target row based on a pixel value and the target value of each pixel on the correction target row, and a correction unit configured to derive a correction coefficient using a pixel value and the target value of the effective pixel and correct the correction target row based on the correction coefficient. It is, therefore, possible to correct detection delay artifacts while reducing the influence of the object.

### Second Embodiment

In the second embodiment, as shown in FIG. 8, an FPD in which dark current electric charges are released at intervals of at least one or more rows will be exemplified. The arrangement of an apparatus and a processing procedure are the same as those in the first embodiment but the contents of target value setting processing in step S201 are different from those in the first embodiment.

In this case, since rows adjacent to both sides of a detection delay artifact occurrence row are normal, a target value setting unit 351 may perform linear prediction based on adjacent pixels on both sides, or use a phase lead or delay low-pass filter with respect to a neighboring pixel by weighing only the normal pixels. Note that since the low-pass filter processes rows except for the detection delay artifact occurrence row, which is equivalent to ½ downsampling, it is designed to perform attenuation at half the Nyquist frequency. Note also that subsequent processes in steps S202 to S204 are the same as those in the first embodiment.

### Third Embodiment

In the third embodiment, a case in which an FPD in which dark current electric charges are released at intervals of at least one or more rows, similarly to the second embodiment, is used, and a plurality of rows are to be read as in FIG. 15 will be described. In this case, the shape of artifacts is such that artifact rows alternately occur, similarly to FIG. 8. All units are the same as those in the second embodiment.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-074862, filed on Mar. 29, 2013, which is hereby incorporated by reference herein in its entirety.

What is claimed is:

1. An image processing apparatus for processing a radiation image obtained from a radiation detector capable of releasing or accumulating electric charges for each row, comprising:
a target value setting unit configured to set a target value of each pixel on a correction target row in the radiation image based on pixel values on a row adjacent to the correction target row;
a pixel selection unit configured to select an effective pixel on the correction target row based on a pixel value and the target value of each pixel on the correction target row; and
a correction unit configured to derive a correction coefficient using a pixel value and the target value of the effective pixel and correct the correction target row based on the correction coefficient.

2. The apparatus according to claim 1, wherein
said correction unit corrects the correction target row using the pixel values and the target values on the correction target row and the correction coefficient.

3. The apparatus according to claim 1, wherein
said pixel selection unit includes
a temporary correction coefficient deriving unit configured to derive temporary correction coefficients based on the pixel values and the target values on the correction target row,
a correction coefficient distribution deriving unit configured to obtain a distribution of the temporary correction coefficients, and
an effective pixel selection unit configured to select the effective pixel based on the distribution of the temporary correction coefficients.

4. The apparatus according to claim 3, wherein
said effective pixel selection unit
calculates a feature value of the temporary correction coefficients based on the distribution of the temporary correction coefficients, and
if at least one of two temporary correction coefficients derived using pairs with other two pixels for each pixel by said temporary correction coefficient deriving unit falls within a threshold range from the feature value, selects the pixel as the effective pixel.

5. The apparatus according to claim 3, wherein
said temporary correction coefficient deriving unit
sorts the respective pixels on the correction target row based on the target values,
creates pairs of pixels by extracting a plurality of pixels having different pixel values for each of the sorted pixels, and
derives the temporary correction coefficients for the pairs for each pixel.

6. The apparatus according to claim 1, wherein
said target value setting unit
derives a gradient between a pixel value of a given pixel on the correction target row and a pixel value of a pixel adjacent to the given pixel,
derives a noise amount from the pixel value of the adjacent pixel, and
sets the target value based on the gradient and the noise amount.

7. The apparatus according to claim 1, wherein
said pixel selection unit determines, as an ineffective pixel, a pixel which has a pixel value not smaller than a saturation pixel value and does not satisfy linearity between the pixel value and a radiation dose of the radiation detector, and excludes the pixel from the effective pixel.

8. An image processing method comprising:
a target value setting step of setting a target value of each pixel on a correction target row in a radiation image based on pixel values on a row adjacent to the correction target row;
a pixel selection step of selecting an effective pixel based on a pixel value and the target value of each pixel on the correction target row; and
a correction step of deriving a correction coefficient using a pixel value and the target value of the effective pixel and correct the correction target row based on the correction coefficient.

9. A non-transitory computer-readable storage medium storing a computer program for causing a computer to execute each step of an image processing method according to claim 8.

10. An image processing apparatus for processing a radiation image obtained from a radiation detector capable of releasing or accumulating electric charges for each row, comprising:
a target value setting unit configured to set a target value of each pixel on a correction target row in the radiation image based on pixel values on a row adjacent to the correction target row; and
a correction unit configured to derive a correction coefficient using a pixel value and the target value of an effective pixel and correct the correction target row based on the correction coefficient,
wherein said target value setting unit performs frequency reduction processing of reducing frequency components.
